(19) United States
(12) Patent Application Publication
(10) Pub. No.: US 2004/0009941 A1
(43) Pub. Date: Jan. 15, 2004

(54) Microspheres and adjuvants for DNA vaccine delivery

(75) Inventors: Mark E. Johnson, Bellevue, WA (US); Sally Mossman, Seattle, WA (US); Tricia Cecil, Bellevue, WA (US); Lawrence Evans, Seattle, WA (US)

Correspondence Address: Attention of Karen S. Canady, Gates & Cooper LLP, Howard Hughes Center, 6701 Center Drive West, Suite 1050, Los Angeles, CA, 90045, US

(73) Assignee: Corixa Corporation

(21) Appl. No.: 10/420,482

(22) Filed: Apr. 22, 2003

Related U.S. Application Data
(62) Division of application No. 09/901,829, filed on Jul. 9, 2001, now abandoned.
(60) Provisional application No. 60/216,604, filed on Jul. 7, 2000.

(51) Int. Cl. A61K 48/00; A61K 31/7024; A61K 9/16; A61K 9/50

(52) U.S. Cl. 514/44; 424/490; 514/42

(57) ABSTRACT

A nucleic acid delivery system that offers, in one system, a combination of high encapsulation efficiency, rapid release kinetics and preservation of DNA in supercoiled form is provided. The nucleic acid delivery system comprises nucleic acid molecules, such as deoxyribonucleic acid (DNA), encapsulated in biodegradable microspheres, and is particularly suited for delivery of DNA vaccines. The invention further provides a method for encapsulating nucleic acid molecules in microspheres. The invention additionally provides a composition comprising nucleic acid molecules encapsulated in microspheres produced by a method of the invention, and a method for delivering a nucleic acid molecule to a subject. The invention further provides an adjuvant for modulating the immunostimulatory efficacy of microspheres encapsulating nucleic acid molecules comprising an aminoalkyl glucosaminide 4-phosphate (AGP). The invention also provides a method for modulating the immunostimulatory efficacy of microspheres encapsulating nucleic acid molecules.

This application is a divisional of U.S. application number 09/901,829, filed Jul. 9, 2001, which claims the benefit of U.S. provisional application No. 60/216,604, filed Jul. 7, 2000, the entire contents of each of which are incorporated herein by reference.

Throughout this application various publications are referenced. The disclosures of these publications in their entireties are hereby incorporated by reference into this application in order to describe more fully the state of the art to which this invention pertains.

### TECHNICAL FIELD OF THE INVENTION

The invention relates to formulations, compositions and methods that can be used for the delivery of vaccines. More particularly, the invention relates to microspheres and adjuvants for more efficient and effective delivery of DNA vaccines.

### BACKGROUND OF THE INVENTION

New vaccines are in development for the prevention, as well as the treatment, of cancers and chronic infectious diseases. The most effective vaccines will likely elicit CTL responses in addition to T-helper responses and antibodies. DNA vaccines have been found to work well in generating CTL responses in mice, although further improvement is needed for use in humans. Attempts to develop microspheres as vehicles for DNA vaccine delivery have been limited by poor encapsulation efficiency, and nicking of the DNA and concomitant loss of supercoiled structure. Efforts to overcome these limitations have produced microspheres whose release kinetics are too slow, resulting in degradation of the DNA while encapsulated.

There remains a need for more efficient and effective means of delivery of DNA vaccines, particularly methods that combine encapsulation efficiency with preservation of DNA supercoiling and rapid release kinetics.

### SUMMARY OF THE INVENTION

The invention provides a nucleic acid delivery system that surprisingly offers, in one system, a combination of high encapsulation efficiency, rapid release kinetics and preservation of DNA in supercoiled form. The nucleic acid delivery system of the invention comprises nucleic acid molecules, such as deoxyribonucleic acid (DNA), encapsulated in biodegradable microspheres. In a preferred embodiment, at least 50% of the DNA in the microspheres comprises supercoiled DNA, and at least 50% of the DNA is released from the microspheres after 7 days at about 37° C. In some embodiments, at least 70% of the DNA is released from the microspheres after 7 days at about 37° C. Preferably, the microspheres have an encapsulation efficiency of at least about 40%. In one embodiment, at least about 90% of the microspheres are about 1 to about 10 μm in diameter. Microspheres in this size range are well-suited to be phagocytosed by antigen-presenting cells, leading to effective T cell stimulation.

The microspheres of the invention preferably comprise a biodegradable polymer, such as poly(lacto-co-glycolide) (PLG), poly(lactide), poly(caprolactone), poly(hydroxybutyrate) and/or copolymers thereof. Alternatively, the microspheres can comprise another wall-forming material. Suitable wall-forming materials include, but are not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers. The nucleic acid delivery system can further comprise an adjuvant, preferably an aminoalkyl glucosaminide 4-phosphate (AGP).

The nucleic acid delivery system of the invention is particularly suited for delivery of DNA vaccines. In preferred embodiments, the DNA encapsulated in the microspheres encodes an antigen associated with cancer or an infectious disease. In one embodiment, the antigen is derived from an endogenous antigen associated with an autoimmune disorder. Examples of cancer include, but are not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, Waldenström's macroglobulinemia, and heavy chain disease. One example of a cancer antigen is Her-2/neu, a breast cancer antigen.

An antigen associated with an infectious disease may be derived from any of a variety of infectious agents, including a pathogen, virus, bacterium, fungus or parasite. Examples of viruses include, but are not limited to, hepatitis type B or type C, influenza, varicella, adenovirus, herpes simplex virus type I or type II, rinderpest, rhinovirus, echovirus, rotavirus, respiratory syncytial virus, papilloma virus, papova virus, cytomegalovirus, echinovirus, arbovirus, huntavirus, coxsachie virus, mumps virus, measles virus, rubella virus, polio virus, human immunodeficiency virus type I or type II. Examples of bacteria include, but are not limited to, M. tuberculosis, mycobacterium, mycoplasma, neisseria and legionella. Examples of parasites include, but are not limited to, rickettsia and chlamydia. One example of an infectious disease antigen is TbH9 (also known as Mtb 39A), a tuberculosis antigen. Other tuberculosis antigens include, but are not limited to, DPV (also known as Mtb8.4), 38-1, Mtb41, Mtb40, Mtb32A, Mtb9.9A, Mtb9.8, Mtb16, Mtb72f, Mtb59f, Mtb88f, Mtb71f, Mtb46f and Mtb31f (“f” indicates that it is a fusion or two or more proteins).

The invention further provides a method for encapsulating nucleic acid molecules in microspheres. The method comprises dissolving a polymer in a solvent to form a polymer solution; adding an aqueous solution containing nucleic acid molecules to the polymer solution to form a primary emulsion; homogenizing the primary emulsion; mixing the primary emulsion with a process medium comprising a stabilizer to form a secondary emulsion; and extracting the solvent from the secondary emulsion to form microspheres encapsulating nucleic acid molecules. Typically, these method steps are carried out on ice, preferably maintaining a temperature that is above freezing and below 37° C. In one embodiment, the solutions and media are maintained at about 2° C. to about 35° C. In another embodiment, the solutions and media are maintained at about 4° C. to about 25° C. Keeping the materials below 37° C. during the primary and secondary emulsion stages of microsphere preparation can reduce nicking of the DNA. Preserving more of the DNA in a supercoiled form facilitates more efficient transfection of cells. The method can further comprise subsequent steps of washing, freezing and lyophilizing the microspheres.

In a preferred embodiment, the polymer comprises PLG. In some embodiments, the PLG can include ester end groups or carboxylic acid end groups, and have a molecular weight of from about 4 kDa to about 120 kDa, or preferably, about 8 kDa to about 65 kDa. The solvent can comprise, for example, dichloromethane, chloroform, or ethylacetate. In some embodiments, the polymer solution further comprises a cationic lipid and/or an adjuvant, such as MPL. Examples of stabilizers include, but are not limited to, carboxymethylcellulose (CMC), polyvinyl alcohol (PVA), polyvinyl pyrrolidone (PVP), or a mixture thereof. The stabilizer can optionally further comprise a cationic lipid. In some embodiments, the stabilizer comprises from about 0 to about 10% of the process medium, or preferably, about 1% to about 5% of the process medium. In some embodiments, the solvent comprises an internal water volume of from about 0.001% to about 0.5%; and/or the aqueous solution comprises an ethanol content of from about 0% to about 75% (v/v).

The nucleic acid molecule preferably comprises DNA. In one embodiment, the aqueous solution comprises about 0.2 to about 12 mg/ml DNA. The aqueous solution can optionally further comprise a stabilizer, such as BSA, HSA, or a sugar, or an adjuvant, such as QS21. In one embodiment, the DNA comprises a plasmid of about 2 kb to about 12 kb, preferably, about 3 kb to about 9 kb.

Preferably, at least 50% of the DNA retains a supercoiled formation through the extraction step, more preferably through any subsequent steps, such as lyophilization. Also preferred is a method wherein the encapsulation efficiency is at least about 40%, and/or wherein the microspheres release at least about 50% of the nucleic acid molecules within about 7 days of contact with the desired delivery environment, such as an aqueous environment at 37° C. In a more preferred embodiment, the microspheres release at least about 50% of the nucleic acid molecules within about 4 days. Also preferred is a method wherein at least about 90% of the microspheres are from about 1 μm to about 10 μm.

The invention additionally provides a composition comprising nucleic acid molecules encapsulated in microspheres produced by a method of the invention. Preferably, the composition further comprises an adjuvant, such as an aminoalkyl glucosaminide 4-phosphate (AGP). Also provided are a method for delivering a nucleic acid molecule to a subject, a method for eliciting an immune response in a subject, and a method for treating and/or protecting against cancer or infectious disease in a subject. These methods comprise administering to the subject a nucleic acid delivery system or a composition of the invention.

The invention further provides an adjuvant for modulating the immunostimulatory efficacy of microspheres encapsulating nucleic acid molecules comprising an aminoalkyl glucosaminide 4-phosphate (AGP). In a preferred embodiment, the AGP comprises an aqueous formulation. Examples of AGP adjuvants include, but are not limited to, 517, 527, 547, 557 and 568. The invention also provides a method for modulating the immunostimulatory efficacy of microspheres encapsulating nucleic acid molecules. The method comprises administering an AGP as an adjuvant to administration of microspheres encapsulating nucleic acid molecules. The AGP can be administered simultaneously with the microspheres, or before or after administration of the microspheres.

### BRIEF DESCRIPTION OF THE FIGURES

FIG. 1 is a scanning electron micrograph illustrating the small and porous nature of DNA microspheres of the invention. In addition to porosity, the microspheres have a high surface area to volume ratio and a short characteristic length of diffusion, facilitating relatively rapid release of encapsulated DNA over 10 days. Bar represents 5 μm; magnification at 3,000×.

FIG. 2 is a graph depicting typical particle size distribution of DNA microspheres formulated in accordance with the invention. The microspheres range from 1-10 μm in diameter, making them well-suited to be phagocytosed by antigen presenting cells.

FIG. 3A is a graph showing encapsulation efficiency as a function of the amount of DNA (in mg) used in a microsphere formulation.

FIG. 3B is a graph showing core-loading of microspheres as a function of the amount of DNA (in mg) used in the formulation. The linear increase in core-loading with increasing DNA amount suggests that encapsulation efficiency may remain essentially constant at approximately 72%. At a core-loading of approximately 1.2%, the microspheres become saturated with DNA such that adding greater amounts of DNA results in lower encapsulation efficiencies.

FIG. 4 shows the results of an agarose gel electrophoresis of unencapsulated DNA (lane 2) and of DNA extracted from PLG microspheres (lanes 3-8). Lane 1 contains molecular weight markers. Minimal nicking (upper bands) of the DNA occurred during microsphere preparation. Specifically, 81% (±3%) of the supercoiled content of the initial DNA was retained after encapsulation and extraction as determined by densitometric analysis. 89% of the naked DNA and 72% of the encapsulated-extracted DNA were in the supercoiled state.

FIG. 5 is a graph showing DNA release kinetics using microspheres of the invention over the course of 10 days. Data are plotted as percent DNA release as a function of time in days. The microsphere formulation released the DNA relatively rapidly, with nearly all of the DNA released by day 10. Such rapid release kinetics are advantageous over slow release (e.g., 30+ days) due in part to the degradation of DNA within microspheres over extended periods of time.

FIG. 6 is a graph showing cytolytic activity of cultured T cells from mice given three 20 μg immunizations at two-week intervals of encapsulated Her-2/neu DNA resuspended in PBS. Cytolytic activity was measured using a standard ⁵¹Cr assay. Data are plotted as percent lysis as a function of effector:target ratio. Mice were immunized i.p. (circles), i.m. (triangles), or s.c. (squares). Filled and open symbols represent specific and non-specific targets, respectively. Each group contained five mice, and average responses are shown. Both i.p. and i.m. immunizations consistently gave better responses, while s.c. immunizations typically resulted in weaker responses.

FIG. 7 is a graph showing cytolytic activity of cultured T cells from mice given a single 10 μg dose of TbH9 DNA i.m. Cytolytic activity was measured using a standard ⁵¹Cr assay. Data are plotted as percent specific lysis as a function of effector:target ratio. Mice received DNA microspheres alone (lower circles), DNA microspheres plus 10 μm of an AGP adjuvant (lines marked 517, 527, 547 and 568), naked DNA (lower squares), or saline (lower triangles). Each group contained four mice, and average responses are shown. Under this sub-optimal immunization schedule (i.e., 1×10 μg immunization with PBS as the buffer), the groups of mice immunized with either naked DNA or with microencapsulated DNA alone failed to generate a substantial CTL response. In contrast, mice immunized with microspheres in combination with AGP-568, 517, or 547 were able to generate strong CTL responses. AGP-527 appeared to be inhibitory in this assay.

FIGS. 8A-D show the molecular structures of aminoalkyl glucosaminide 4-phosphates (AGPs) evaluated in conjunction with DNA microspheres. These synthetic molecules were prepared using an enantioselective process.

FIG. 9 is a graph showing cytolytic activity of cultured T cells from mice given a single 10 μg dose of TbH9 DNA resuspended in either PBS (triangles) or sodium chloride free, isotonic phosphate buffer (circles). Squares represent mice immunized with saline. Cytolytic activity was measured using a standard ⁵¹Cr assay. Data are plotted as percent specific lysis as a function of effector:target ratio. Each group contained four mice, and average responses are shown. Under this sub-optimal immunization schedule (i.e., 1×10 μg immunization), the group of mice immunized with microencapsulated DNA dispersed in PBS failed to generate a substantial CTL response. In contrast, mice immunized with microspheres dispersed in isotonic phosphate buffer (i.e., sodium chloride free) generated strong CTL responses.

FIG. 10 is a bar graph showing IFN-gamma secretion (in pg/ml) in response to in vitro stimulation with recombinant TbH9, assayed using splenocytes harvested from mice 3-4 weeks following immunization with TbH9 DNA encapsulated in PLG microspheres with AGP.

FIG. 11 is a graph showing mean CTL activity after a single in vitro stimulation with EL-4 cells stably expressing the TbH9 gene of splenocytes harvested from mice immunized with TbH9 DNA encapsulated in PLG microspheres to which AGP was added. The graph shows mean specific lysis as a function of effector:target ratio for immunization conditions including saline (closed diamonds), naked DNA (dark squares), DNA-PLG (lower triangles), and DNA-PLG plus AGP-517 (light X's), 522 (asterisks), 525 (circles), 527 (+'s), 529 (dashed line), 540 (−'s), 544 (open diamonds), 547 (light squares), 557 (upper triangles), or 578 (dark X's).

FIG. 12A shows mean CTL activity after a second in vitro stimulation of splenocytes from mice immunized with TbH9 DNA-PLG alone (open squares), with AGP-527 (closed squares), 544 (dark diamonds), 557 (closed circles), or with naked DNA (open circles) or saline (triangles).

FIG. 12B shows mean CTL activity after a second in vitro stimulation of splenocytes from mice immunized with TbH9 DNA-PLG with AGP-517 (closed squares), 547 (dark diamonds), 568 (light triangles), or with naked DNA (X's).

FIGS. 13A-B are graphs showing serum antibody titers to TbH9 of Rhesus macaque monkeys four weeks after a 3^{rd} immunization with TbH9, encapsulated in microspheres and administered intramuscularly (FIG. 13A), or delivered as naked DNA via intradermal or intramuscular routes (FIG. 13B). The four lines depicted in each graph represent individual subjects.

FIG. 14 is a bar graph showing antigen-induced gamma interferon (IFN-γ) production from monkey PBMC at 4 weeks after a 3^{rd} immunization with saline, recombinant TbH9 (rTbH9), naked DNA encoding TbH9 or microspheres encapsulating DNA encoding TbH9. Individual bars represent individual subjects.

FIGS. 15A-B are graphs showing monkey CTL response at two months after a 3^{rd} immunization with microencapsulated (FIG. 15A) or naked (FIG. 15B) DNA encoding TbH9. Percent specific lysis is plotted as a function of effector:target ratio. Circles represent TbH9 target cells. Control targets include non-infected cells (squares) and, as non-specific targets, EGFP (a fluorescent protein) cells (triangles).

### DETAILED DESCRIPTION OF THE INVENTION

The invention provides a nucleic acid delivery system that surprisingly offers, in one system, a combination of high encapsulation efficiency, rapid release kinetics and preservation of DNA in supercoiled form. The nucleic acid delivery system of the invention comprises nucleic acid molecules, such as deoxyribonucleic acid (DNA), encapsulated in biodegradable microspheres. Microspheres prepared in accordance with the invention have been shown to release more than 33% of their contents after 48 hours in vitro at 37° C., more than 50% after 4 days, and more than 70% after 7 days. In addition, these microspheres have an encapsulation efficiency of about 40 to about 80%, while retaining a high ratio of supercoiled to nicked DNA. The microspheres of the invention are about 1 to about 10 μm in diameter. Microspheres in this size range are well-suited to be phagocytosed by antigen-presenting cells, leading to effective T cell stimulation. The nucleic acid delivery system of the invention can be used to deliver nucleic acid molecules encoding one or more antigens of interest for the elicitation of an immune response in a subject.

The invention further provides an adjuvant for modulating the immunostimulatory efficacy of microspheres encapsulating nucleic acid molecules. The adjuvant comprises an aminoalkyl glucosaminide 4-phosphate (AGP), which provides a strong cellular immune response to an antigen encoded by DNA encapsulated in microspheres. The invention also provides a method for modulating the immunostimulatory efficacy of microspheres encapsulating nucleic acid molecules. The method comprises administering an AGP as an adjuvant to administration of microspheres encapsulating nucleic acid molecules.

Definitions

All scientific and technical terms used in this application have meanings commonly used in the art unless otherwise specified. As used in this application, the following words or phrases have the meanings specified.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides.

As used herein, “immune response” includes the production of antibodies, production of immunomodulators such as IFN-γ, and induction of CTL activity. The elicitation of an immune response includes the initiation, stimulation or enhancement of an immune response.

As used herein, to “prevent” or “protect against” a condition or disease means to hinder, reduce or delay the onset or progression of the condition or disease.

As used herein, “antigen-presenting cell” or “APC” means a cell capable of handling and presenting antigen to a lymphocyte. Examples of APCs include, but are not limited to, macrophages, Langerhans-dendritic cells, follicular dendritic cells, B cells, monocytes, fibroblasts and fibrocytes. Dendritic cells are a preferred type of antigen presenting cell. Dendritic cells are found in many non-lymphoid tissues but can migrate via the afferent lymph or the blood stream to the T-dependent areas of lymphoid organs. In non-lymphoid organs, dendritic cells include Langerhans cells and interstitial dendritic cells. In the lymph and blood, they include afferent lymph veiled cells and blood dendritic cells, respectively. In lymphoid organs, they include lymphoid dendritic cells and interdigitating cells.

As used herein, “modified” to present an epitope refers to antigen-presenting cells (APCs) that have been manipulated to present an epitope by natural or recombinant methods. For example, the APCs can be modified by exposure to the isolated antigen, alone or as part of a mixture, peptide loading, or by genetically modifying the APC to express a polypeptide that includes one or more epitopes.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, Chapter 43, 14th Ed., Mack Publishing Co, Easton Pa. 18042, USA).

As used herein, “adjuvant” includes those adjuvants commonly used in the art to facilitate the stimulation of an immune response. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mich.); Merck Adjuvant 65 (Merck and Company, Inc., Rahway, N.J.); AS-2 (Smith-Kline Beecham); QS-21 (Aquilla); MPL™ immunostimulant or 3d-MPL (Corixa Corporation); LEIF; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; muramyl tripeptide phosphatidyl ethanolamine or an immunostimulating complex, including cytokines (e.g., GM-CSF or interleukin-2, -7 or -12) and immunostimulatory DNA sequences. In some embodiments, such as with the use of a polynucleotide vaccine, an adjuvant such as a helper peptide or cytokine can be provided via a polynucleotide encoding the adjuvant.

As used herein, “a” or “an” means at least one, unless clearly indicated otherwise.

Nucleic Acid Delivery Systems

The invention provides a nucleic acid delivery system comprising deoxyribonucleic acid (DNA) encapsulated in biodegradable microspheres. In a preferred embodiment, at least 50% of the DNA in the microspheres comprises supercoiled DNA, and at least 50% of the DNA is released from the microspheres after 7 days at about 37° C. In some embodiments, at least 70% of the DNA is released from the microspheres after 7 days at about 37° C. Preferably, the microspheres have an encapsulation efficiency of at least about 40%. In one embodiment, at least about 90% of the microspheres are about 1 to about 10 μm in diameter. Microspheres in this size range are well-suited to be phagocytosed by antigen-presenting cells, leading to effective T cell stimulation.

The microspheres of the invention preferably comprise a biodegradable polymer, such as poly(lacto-co-glycolide) (PLG), poly(lactide), poly(caprolactone), poly(hydroxybutyrate) and/or copolymers thereof. Alternatively, the microspheres can comprise another wall-forming material. Suitable wall-forming materials include, but are not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like;, poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers. The nucleic acid delivery system can further comprise an adjuvant, preferably an aminoalkyl glucosaminide 4-phosphate (AGP).

Microsphere Formulation

The invention provides a method for encapsulating nucleic acid molecules in microspheres. The method comprises dissolving a polymer in a solvent to form a polymer solution; adding an aqueous solution containing nucleic acid molecules to the polymer solution to form a primary emulsion; homogenizing the primary emulsion; mixing the primary emulsion with a process medium comprising a stabilizer to form a secondary emulsion; and extracting the solvent from the secondary emulsion to form microspheres encapsulating nucleic acid molecules. Typically, these method steps are carried out on ice, preferably maintaining a temperature that is above freezing and below 37° C. In one embodiment, the solutions and media are maintained at about 2° C. to about 35° C. In another embodiment, the solutions and media are maintained at about 4° C. to about 25° C. Keeping the materials below 37° C. during the primary and secondary emulsion stages of microsphere preparation can reduce nicking of the DNA. Preserving mote of the DNA in a supercoiled form facilitates more efficient transfection of cells. The method can further comprise subsequent steps of washing, freezing and lyophilizing the microspheres.

In a preferred embodiment, the polymer comprises PLG. In some embodiments, the PLG can include ester end groups or carboxylic acid end groups, and have a molecular weight of from about 4 kDa to about 120 kDa, or preferably, about 8 kDa to about 65 kDa. The solvent can comprise, for example, dichloromethane, chloroform, or ethylacetate. In some embodiments, the polymer solution further comprises a cationic lipid and/or an adjuvant, such as MPL. Examples of stabilizers include, but are not limited to, carboxymethylcellulose (CMC), polyvinyl alcohol (PVA), polyvinyl pyrrolidone (PVP), or a mixture thereof. The stabilizer can optionally further comprise a cationic lipid. In some embodiments, the stabilizer comprises from about 0 to about 10% of the process medium, or preferably, about 1% to about 5% of the process medium. In some embodiments, the solvent comprises an internal water volume of from about 0.001% to about 0.5%; and/or the aqueous solution comprises an ethanol content of from about 0% to about 75% (v/v).

The nucleic acid molecule preferably comprises DNA. In one embodiment, the aqueous solution comprises about 0.2 to about 12 mg/ml DNA. The aqueous solution can optionally further comprise a stabilizer, such as BSA, HSA, or a sugar, or an adjuvant, such as QS21. In one embodiment, the DNA comprises a plasmid of about 2 kb to about 12 kb, preferably, about 3 kb to about 9 kb.

Preferably, at least 50% of the DNA retains a supercoiled formation through the extraction step, more preferably through any subsequent steps, such as lyophilization. Also preferred is a method wherein the encapsulation efficiency is at least about 40%, and/or wherein the microspheres release at least about 50% of the nucleic acid molecules within about 7 days of contact with the desired delivery environment, such as an aqueous environment at 37° C. In a more preferred embodiment, the microspheres release at least about 50% of the nucleic acid molecules within about 4 days. Also preferred is a method wherein at least about 90% of the microspheres are from about 1 μm to about 10 μm.

Because water-soluble agents, such as nucleic acid molecules, do not diffuse through hydrophobic wall-forming materials such as the lactide/glycolide copolymers, pores must be created in the microsphere membrane to allow these agents to diffuse out for controlled-release applications. Several factors will affect the porosity obtained. The amount of agent that is encapsulated affects the porosity of microspheres. Obviously, higher-loaded microspheres (i.e., greater than about 20 wt. %, and preferably between 20 wt. % and 80 wt. %) will be more porous than microspheres containing smaller amounts of agent (i.e., less than about 20 wt. %) because more regions of drug are present throughout the microspheres. The ratio of agent to wall-forming material that can be incorporated into the microspheres can be as low as 0.1% to as high as 80%.

The solvent used to dissolve the wall-forming material will also affect the porosity of the membrane. Microspheres prepared from a solvent such as ethyl acetate will be more porous than microspheres prepared from chloroform. This is due to the higher solubility of water in ethyl acetate than in chloroform. More specifically, during the emulsion step, no solvent is removed from the microdroplets because the process medium is saturated with solvent. Water, however, can dissolve in the solvent of the microdroplets during the emulsion step of the process. By selecting the appropriate solvent or cosolvents, the amount of continuous process medium that will dissolve in the microdroplets can be controlled, which will affect the final porosity of the membrane and the internal structure of the microspheres.

Another factor that will affect the porosity of the membrane is the initial concentration of the wall material/excipient in the solvent. High concentrations of wall material in the solvent result in less porous membranes than do low-concentrations of wall material/excipient. Also, high concentrations of wall material/excipient in the solvent improve the encapsulation efficiency of water-soluble compounds because the viscosity of the solution is higher. Generally, the concentration of wall-forming material/excipient in the solvent will range from about 3% to about 40%, depending on the physical/chemical properties of the wall material/excipient such as the molecular weight of the wall-forming material and the solvent used.

Compositions

The invention provides compositions that are useful for delivering nucleic acid molecules. The nucleic acid molecules can include those encoding antigens associated with cancer or infectious disease, providing compositions for treating and preventing cancer or infectious disease. In one embodiment, the composition is a pharmaceutical composition. The composition can comprise a therapeutically or prophylactically effective amount of a polynucleotide, recombinant virus, APC or immune cell that encodes or presents one or more antigens associated with cancer or infectious disease. An effective amount is an amount sufficient to elicit or augment an immune response, e.g., by activating T cells. One measure of the activation of T cells is a cytotoxicity assay or an interferon-gamma release assay, as described in the examples below. In some embodiments, the composition is a vaccine.

In some embodiments, the condition to be treated or prevented is cancer or a precancerous condition (e.g., hyperplasia, metaplasia, dysplasia). Examples of cancer include, but are not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, Waldenström's macroglobulinemia, and heavy chain disease.

In some embodiments, the condition to be treated or prevented is an infectious disease. Examples of infectious disease include, but are not limited to, infection with a pathogen, virus, bacterium, fungus or parasite. Examples of viruses include, but are not limited to, hepatitis type B or type C, influenza, varicells, adenovirus, herpes simplex virus type I or type II, rinderpest, rhinovirus, echovirus, rotavirus, respiratory syncytial virus, papilloma virus, papova virus, cytomegalovirus, echinovirus, arbovirus, huntavirus, coxsachie virus, mumps virus, measles virus, rubella virus, polio virus, human immunodeficiency virus type I or type II. Examples of bacteria include, but are not limited to, M. tuberulosis, mycobacterium, mycoplasma, neisseria and legionella. One example of an M. tuberculosis antigen is TbH9 (also known as Mtb 39A), a tuberculosis antigen. Other tuberculosis antigens include, but are not limited to, DPV (also known as Mtb8.4), 38-1, Mtb41, Mtb40, Mtb32A, Mtb9.9A, Mtb9.8, Mtb16, Mtb72f, Mtb59f, Mtb88f, Mtb71f, Mtb46f and Mtb31f (“f” indicates that it is a fusion or two or more proteins). Examples of parasites include, but are not limited to, rickettsia and chlamydia.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The composition of the invention can further comprise one or more adjuvants. Examples of adjuvants include, but are not limited to, helper peptide, alum, Freund's, muramyl tripeptide phosphatidyl ethanolamine or an immunostimulating complex, including cytokines. In some embodiments, such as with the use of a polynucleotide vaccine, an adjuvant such as a helper peptide or cytokine can be provided via a polynucleotide encoding the adjuvant. A preferred adjuvant is AGP.

Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive.

Biodegradable microspheres (e.g., polylactate polyglycolate) for use as carriers are disclosed, for example, in U.S. Pat. Nos. 4,897,268; 5,075,109; 5,928,647; 5,811,128; 5,820,883; 5,853,763; 5,814,344; 5,407,609; and 5,942,252; the disclosures of each of which are incorporated herein by reference. In particular, these patents, such as U.S. Pat. Nos. 4,897,268 and 5,407,609, describe the production of biodegradable microspheres for a variety of uses, but do not teach the optimization of microsphere formulation and characteristics for DNA delivery.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

Adjuvants

The invention further provides adjuvants for use with DNA vaccines, particularly for use with DNA vaccines encapsulated in biodegradable microspheres. Such adjuvants comprise an aminoalkyl glucosaminide 4-phosphate (AGP), such as those described in pending U.S. patent application Ser. Nos. 08/853,826 and 09/074,720, the disclosures of which are incorporated herein by reference in their entireties.

Compositions of the invention can include an AGP adjuvant and/or additional adjuvants. Most adjuvants contain a substance designed to protect the antigen from rapid catabolism, such as aluminum hydroxide or mineral oil, and a stimulator of immune responses, such as lipid A, Bortadella pertussis or Mycobaterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes biodegradable microspheres; monophosphoryl lipid A and quil A. Cytokines, such as GM CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, IL-2 and IL-12) tend to favor the induction of cell mediated immune responses to an administered antigen. In contrast, high levels of Th2-type cytokines (e.g., IL-4, IL-5, IL-6, IL-10 and TNF-β) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, 1989, Ann. Rev. Immunol. 7:145-173.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Ribi ImmunoChem Research Inc. (Hamilton, Mont.) (see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555. Another preferred adjuvant is a saponin, preferably QS21, which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprises an oil-in-water emulsion and tocopherol. A particularly potent adjuvant formulation involving QS21, 3D-MPL and tocopherol in an oil-in-water emulsion is described in WO 95/17210. Another adjuvant that may be used is AS-2 (Smith-Kline Beecham). Any vaccine provided herein may be prepared using well known methods that result in a combination of antigen, immune response enhancer and a suitable carrier or excipient.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations, may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Methods

The invention provides a method for delivering a nucleic acid molecule to a subject. The invention additionally provides a method for eliciting an immune response in a subject, and a method for treating and/or protecting against cancer or infectious disease in a subject. The method comprises administering to the subject a nucleic acid delivery system or a composition of the invention. Administration can be performed as described above. In one embodiment, the cancer is breast cancer. In this embodiment, a preferred nucleic acid delivery system comprises a nucleic acid molecule encoding the breast cancer antigen, her2/neu. In another embodiment, the infectious disease is tuberculosis. In this embodiment, a preferred nucleic acid delivery system comprises a nucleic acid molecule encoding the tuberculosis antigen, TbH9.

The invention also provides a method for modulating the immunostimulatory efficacy of microspheres encapsulating nucleic acid molecules. The method comprises administering an AGP as an adjuvant to administration of microspheres encapsulating nucleic acid molecules. The AGP can be administered simultaneously with the microspheres, or before or after administration of the microspheres. The AGP may be encapsulated with the DNA inside the microspheres, included in a composition with the microspheres, or administered in a separate composition from the microspheres. In a typical embodiment of the method of the invention, the AGP enhances the immune response elicited by microspheres encapsulating nucleic acid molecules.

A delivery vehicle of the invention may be employed to facilitate production of an antigen-specific immune response that targets cancerous or infected cells. Certain preferred embodiments of the present invention use dendritic cells or progenitors thereof as antigen-presenting cells (APCs). Dendritic cells are highly potent APCs (Banchereau and Steinman, Nature 392:245-251, 1998) and have been shown to be effective as a physiological adjuvant for eliciting prophylactic or therapeutic immunity (see Timmerman and Levy, Ann. Rev. Med. 50:507-529, 1999). In general, dendritic cells may be identified based on their typical shape (stellate in situ, with marked cytoplasmic processes (dendrites) visible in vitro) and based on the lack of differentiation markers of B cells (CD19 and CD20), T cells (CD3), monocytes (CD14) and natural killer cells (CD56), as determined using standard assays. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (Zitvogel et al., 1998, Nature Med. 4:594-600).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor, mannose receptor and DEC-205 marker. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80 and CD86). APCs may be combined with a polynucleotide encapsulated in a microsphere of the invention such that the APCs can take up the DNA and express the polypeptide, or an immunogenic portion thereof, which is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., 1997, Immunology and Cell Biology 75:456-460. Antigen loading of dendritic cells may be achieved by incubating dendritic cells or progenitor cells with the encapsulated DNA or RNA. A dendritic cell may be pulsed with an immunological partner that provides T cell help (e.g., a carrier molecule).

Administration of the Compositions

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals. Preferably, the patients or subjects are human.

Compositions are typically administered in vivo via parenteral (e.g. intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue. Intramuscular administration is preferred.

The dose administered to a patient, in the context of the present invention should be sufficient to effect a beneficial therapeutic response in the patient over time, or to inhibit infection or disease due to infection. Thus, the composition is administered to a patient in an amount sufficient to elicit an effective immune response to the specific antigens and/or to alleviate, reduce, cure or at least partially arrest or prevent symptoms and/or complications from the disease or infection. An amount adequate to accomplish this is defined as a “therapeutically effective dose.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of diseases, the physician needs to evaluate the production of an immune response against the pathogen, progression of the disease, and any treatment-related toxicity.

Compositions comprising immune cells are preferably prepared from immune cells obtained from the subject to whom the composition will be administered. Alternatively, the immune cells can be prepared from an HLA-compatible donor. The immune cells are obtained from the subject or donor using conventional techniques known in the art, exposed to APCs modified to present an epitope of the invention, expanded ex vivo, and administered to the subject. Protocols for ex vivo therapy are described in Rosenberg et al., 1990, New England J. Med. 9:570-578.

Immune cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to enrich and rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., 1997, Immunological Reviews 157:177).

Administration by many of the routes of administration described herein or otherwise known in the art may be accomplished simply by direct administration using a needle, catheter or related device, at a single time point or at multiple time points.

### EXAMPLES

The following examples are presented to illustrate the present invention and to assist one of ordinary skill in making and using the same. The examples are not intended in any way to otherwise limit the scope of the invention.

### Example 1

DNA Encapsulated in PLG Microspheres Generates CTL Responses

This example describes the formulation of a DNA PLG microsphere with desirable in vitro characteristics. Specifically, 1-10 μm diameter microspheres which were able to release their DNA contents over the course of a week were prepared using a process that resulted in a high encapsulation efficiency (60-80%) and high rate of retention of the DNA supercoiled state (70%). Once these microspheres were found to generate cytotoxic T-lymphocyte (CTL) responses in mice to plasmids encoding protein antigens for both an infectious disease (tuberculosis) and cancer, a series of experiments were performed to elucidate the factors responsible for generating the strongest and most consistent CTL responses. Intramuscular and intraperitoneal immunizations were the most efficacious routes of immunization. The microsphere resuspension buffer was also found to be an important parameter, with PBS inhibiting CTL responses relative to salt free buffer. In addition, several aminoalkyl glucosaminide 4-phosphates (AGPs) adjuvants were found to enhance CTL responses in conjunction with these DNA microspheres.

Materials & Methods

PLG microspheres containing DNA encoding antigenic proteins were prepared using variations on the double emulsion technique (J. H. Eldridge et al. Mol Immunol, 28:287-294, 1991; S. Cohen et al. Pharm Res, 8:713-720, 1991). Specifically, plasmid DNA (10-30 mgs) in Tris-EDTA buffer (10 mM; pH 8), 0.38 ml ethanol were combined and brought up to a volume of 5.1 ml using Tris EDTA buffer (10 mM; pH 8). This is the internal (water) phase. 1200 mg of poly(lactide-co-glycolide) polymer was dissolved in 13.9 ml of dichloromethane (DCM) and put on ice. The internal aqueous phase was added to the PLG solution and mixed in a 30 ml syringe while still on ice using a Polytron tissue homogenizer for 20 seconds to form the primary emulsion (water-in-oil). The secondary emulsion was prepared by adding the primary emulsion to a beaker containing 280 ml of 1.4% carboxymethylcellulose (w/v), or process medium, on ice, and mixing with a Silverson mixer for 75 seconds at 4500 rpm while still on ice. The secondary emulsion was diluted with approximately five liters of MiliQ water, and mixed using an overhead stirrer for approximately 20 minutes in order to extract dichloromethane from, and to harden, the microspheres.

The resulting microspheres were washed 2-3 times using MiliQ water and centrifugation. After washing, mannitol was added tot he concentrated microspheres, which were frozen and lyophilized. Lyophilized microspheres were then assayed for their size distribution, DNA content (core-loading; from this value, the encapsulation efficiency was calculated), release kinetics, and the supercoiled content of the encapsulated DNA.

Particle sizing was performed using MIE light scattering. Core-loadings were determined by dissolving the microspheres in methylene chloride and extracting the DNA with aqueous buffer. DNA concentrations were then measured using the PicoGreen fluorescence assay. The forms of the plasmids were determined through digital u.v. image analysis of agarose gels. Two plasmids were used in this study, one encoding a tuberculosis antigen, TbH9, and the other encoding the breast cancer antigen Her-2/neu.

Mice were immunized with DNA microspheres dispersed in aqueous buffer. Several routes of administration, including i.m., i.p., and s.c., were examined by giving the mice 3×20 μg immunizations two weeks apart. Previous work had shown that multiple immunizations and higher doses of encapsulated DNA yielded stronger CTL responses. The combination of microspheres with select aminoalkyl glucosaminide 4-phosphates adjuvants was investigated by using a sub-optimal immunziation schedule—a single 10 μg dose of encapsulated DNA dispersed in PBS—along with 10 μg of adjuvant. Lastly, the effect of the resuspension buffer was examined by administering to mice a single 10 μg dose of encapsulated DNA dispersed in either PBS or sodium chloride free phosphate buffer (PB).

CTL responses were measured using spleen cells harvested from the mice. CTL lines were generated by culture of immune spleen cells with APC lines transfected with the antigen of interest. Lines were stimulated weekly and CTL activity was assessed in a standard 51Chromium release assay 6 days after the in vitro stimulation.

Results

The process resulted in microspheres that were small (about 1-10 μm in diameter), with rapid release kinetics, high encapsulation efficiency (40-80%), and good retention of supercoiled DNA. More than 33% of the microsphere contents were released after 48 hours in vitro at 37° C.; more than 50% were released after four days; and more than 70% after 7 days. The ratio of supercoiled-to-nicked DNA for the plasmid extracted from the microspheres was more than 50% of the ratio of the input DNA.

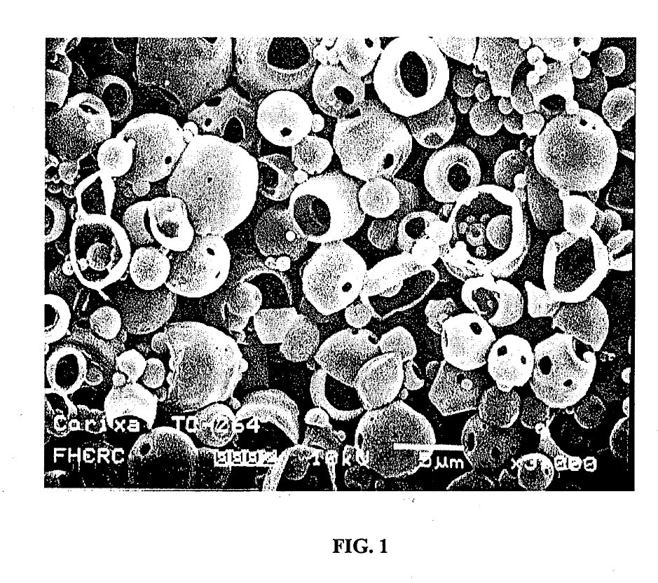

FIG. 1 is a scanning electron micrograph illustrating the small and porous nature of DNA microspheres of the invention. In addition to porosity, the microspheres have a high surface area to volume ratio and a short characteristic length of diffusion, facilitating relatively rapid release of encapsulated DNA over 10 days. Bar represents 5 μm; magnification at 3,000×.

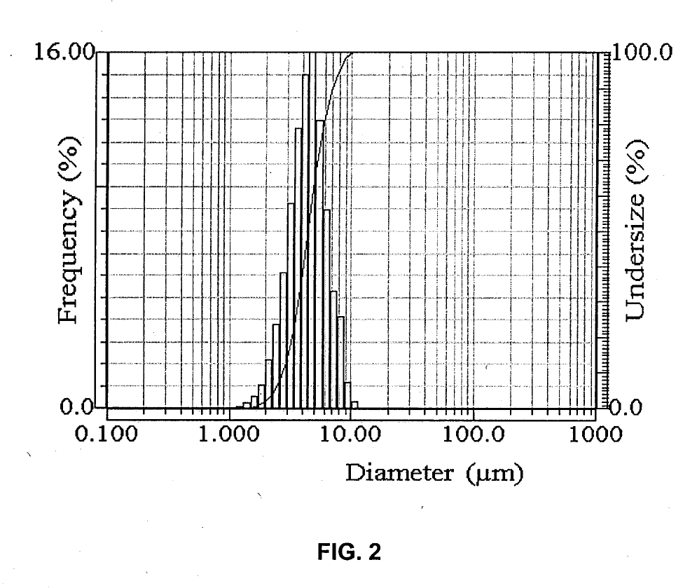

FIG. 2 is a graph depicting typical particle size distribution of DNA microspheres formulated in accordance with the invention. The microspheres range from 1-10 μm in diameter, making them well-suited to be phagocytosed by antigen presenting cells.

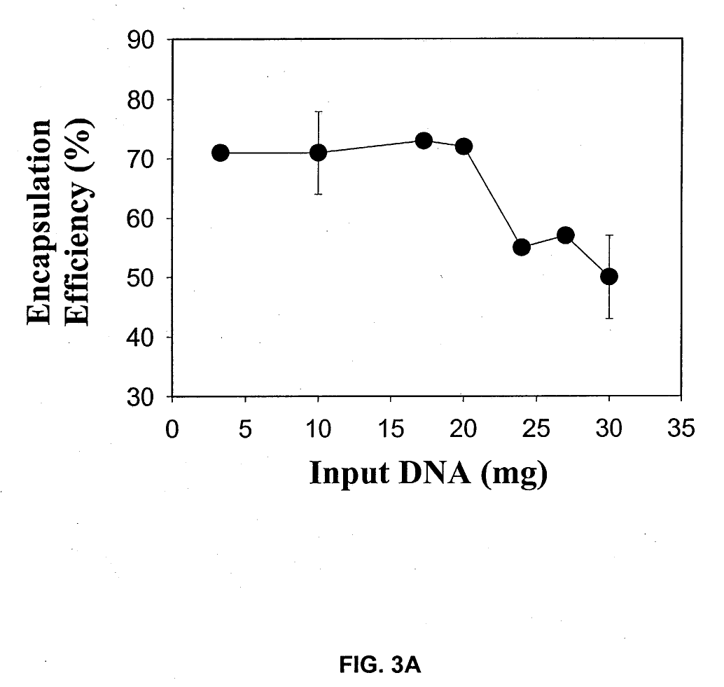

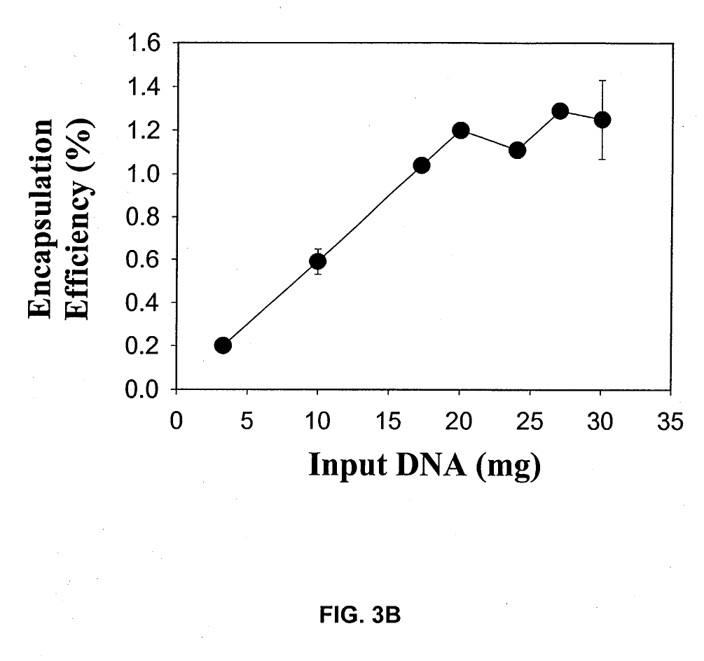

FIG. 3A is a graph showing encapsulation efficiency as a function of the amount of DNA (in mg) used in a microsphere formulation.

FIG. 3B is a graph showing core-loading of microspheres as a function of the amount of DNA (in mg) used in the formulation. The linear increase in core-loading with increasing DNA amount suggests that encapsulation efficiency may remain essentially constant at approximately 72%. At a core-loading of approximately 1.2%, the microspheres become saturated with DNA such that adding greater amounts of DNA results in lower encapsulation efficiencies.

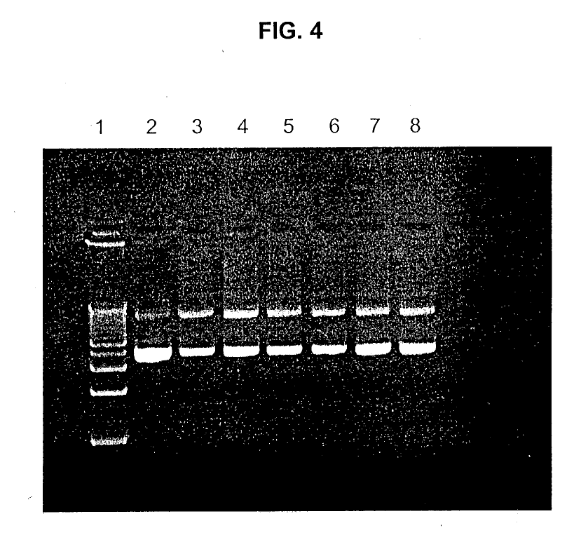

FIG. 4 shows the results of an agarose gel electrophoresis of unencapsulated DNA (lane 2) and of DNA extracted from PLG microspheres (lanes 3-8). Lane 1 contains molecular weight markers. Minimal nicking (upper bands) of the DNA occurred during microsphere preparation. Specifically, 81% (±3%) of the supercoiled content of the initial DNA was retained after encapsulation and extraction as determined by densitometric analysis. 89% of the naked DNA and 72% of the encapsulated-extracted DNA were in the supercoiled state.

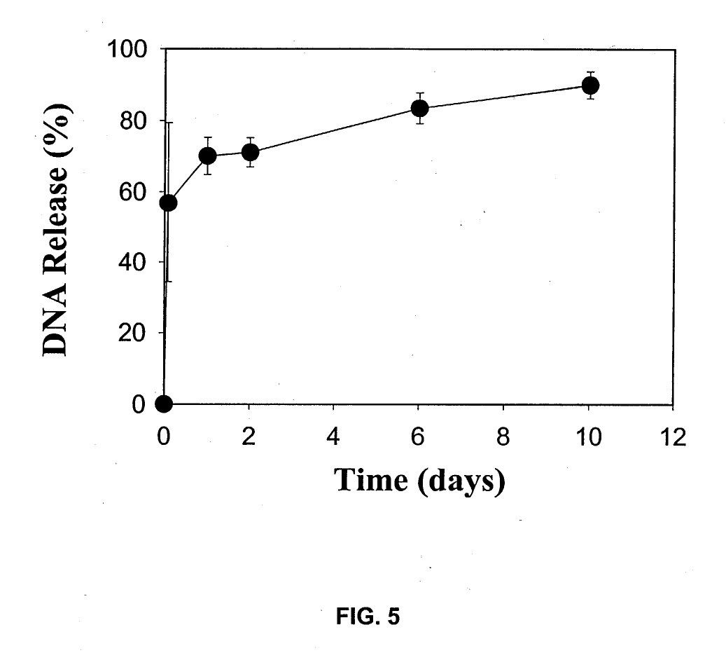

FIG. 5 is a graph showing DNA release kinetics using microspheres of the invention over the course of 10 days. Data are plotted as percent DNA release as a function of time in days. The microsphere formulation released the DNA relatively rapidly, with nearly all of the DNA released by day 10. Such rapid release kinetics are advantageous over slow release (e.g., 30+ days) due in part to the degradation of DNA within microspheres over extended periods of time.

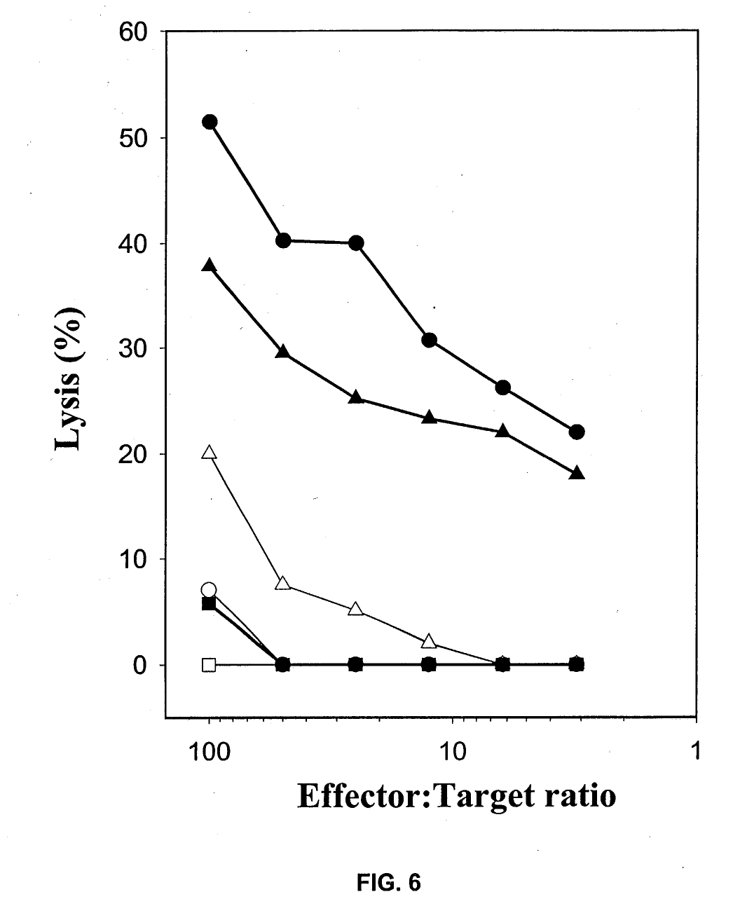

FIG. 6 is a graph showing cytolytic activity of cultured T cells from mice given three 20 μg immunizations at two-week intervals of encapsulated Her-2/neu DNA resuspended in PBS. Cytolytic activity was measured using a standard ⁵¹Cr assay. Data are plotted as percent lysis as a function of effector:target ratio. Mice were immunized i.p. (circles), i.m. (triangles), or s.c. (squares). Filled and open symbols represent specific and non-specific targets, respectively. Each group contained five mice, and average responses are shown. Both i.p. and i.m. immunizations consistently gave better responses, while s.c. immunizations typically resulted in weaker responses.

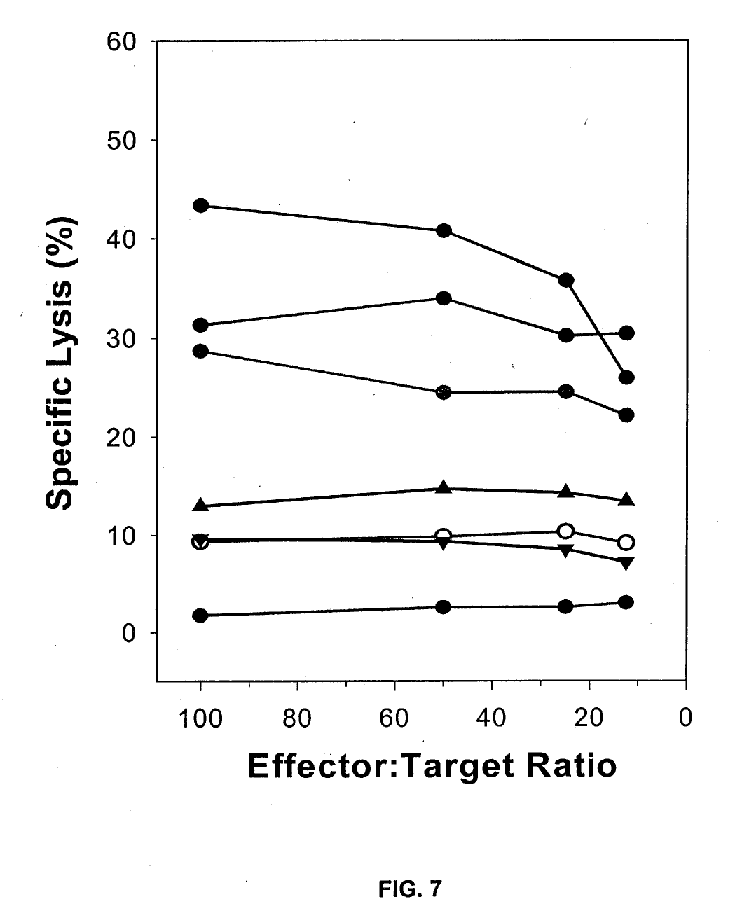

FIG. 7 is a graph showing cytolytic activity of cultured T cells from mice given a single 10 μg dose of TbH9 DNA i.m. Cytolytic activity was measured using a standard ⁵¹Cr assay. Data are plotted as percent specific lysis as a function of effector:target ratio. Mice received DNA microspheres alone (lower circles), DNA microspheres plus 10 μg of an AGP adjuvant (lines marked 517, 527, 547 and 568), naked DNA (lower squares), or saline (lower triangles). Each group contained four mice, and average responses are shown. Under this sub-optimal immunization schedule (i.e., 1×10 μg immunization with PBS as the buffer), the groups of mice inmunized with either naked DNA or with microencapsulated DNA alone failed to generate a substantial CTL response. In contrast, mice immunized with microspheres in combination with AGP-568, 517, or 547 were able to generate strong CTL responses. AGP-527 appeared to be inhibitory in this assay.

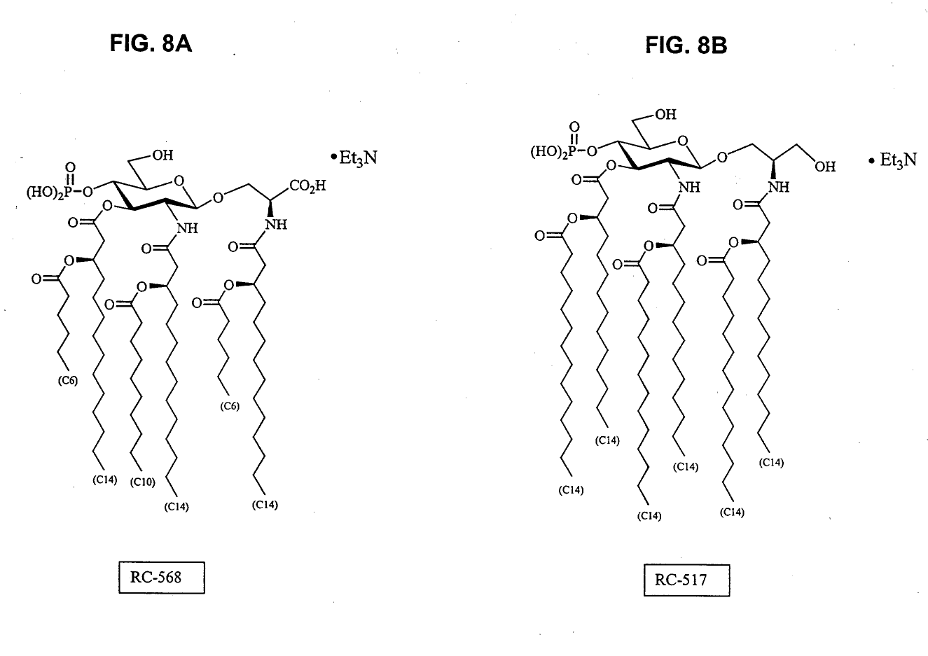

FIG. 8 shows the molecular structures of aminoalkyl glucosaminide 4-phosphates (AGPs) evaluated in conjunction with DNA microspheres. These synthetic molecules were prepared using an enantioselective process.

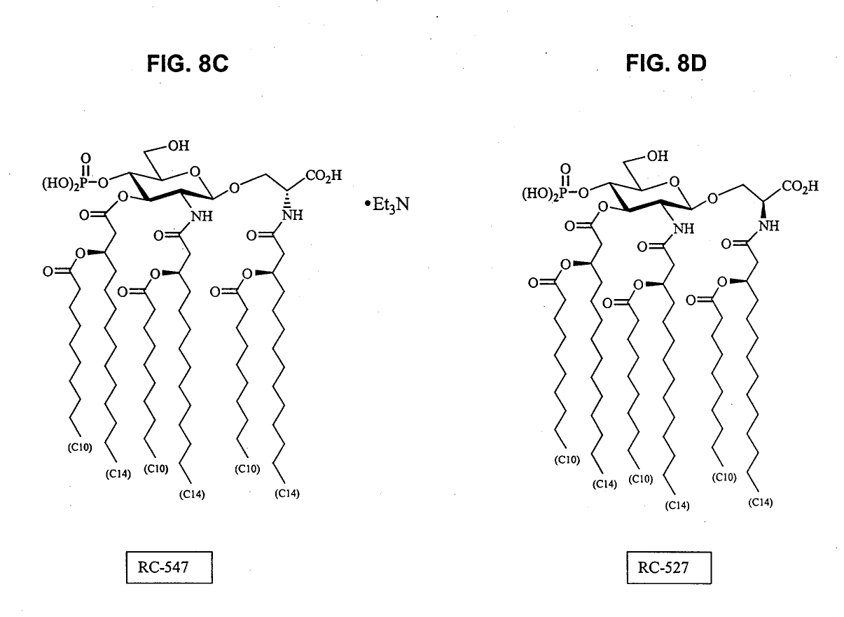

FIG. 9 is a graph showing cytolytic activity of cultured T cells from mice given a single 10 μg dose of TbH9 DNA resuspended in either PBS (triangles) or sodium chloride free, isotonic phosphate buffer (circles). Squares represent mice immunized with saline. Cytolytic activity was measured using a standard ⁵¹Cr assay. Data are plotted as percent specific lysis as a function of effector:target ratio. Each group contained four mice, and average responses are shown. Under this sub-optimal immunization schedule (i.e., 1×10 μg immunization), the group of mice immunized with microencapsulated DNA dispersed in PBS failed to generate a substantial CTL response. In contrast, mice immunized with microspheres dispersed in isotonic phosphate buffer (i.e., sodium chloride free) generated strong CTL responses.

Numerous variations to the process described above were made, without substantively changing the basic properties of the microspheres. These variations include:

Internal Water Phase: Ethanol content was varied from 0% up to 75% (v/v). Volume was varied from 0.1 ml up to 6.6 ml. Adjuvants were added, including QS21. Stabilizers were added, including bovine serum albumin (BSA).

DNA: The amount of DNA was varied from 1 mg up to 60 mg. The concentration of DNA in the internal water phase was varied from 0.2 up to 12 mg/ml. The size of the plasmid was varied between about 3 kb to about 9 kb. The antigen encoded by the plasmid was also varied, such as her-2/neu and TbH9.

Polymer: The end group on the PLG polymer was varied between ester end groups and carboxylic acid end groups. The molecular weight of the PLG polymer was varied from about 8 kDa up to 65 kDa. A cationic lipid (DOTAP) was, in some cases, added to the polymer solution, and varied from 0.5 to 5 mg. The amount of PLG polymer was varied between 150 and 3000 mg. Adjuvants, including MPL, were added to the polymer solution.

Solvent: The solvent was varied between dichloromethane, chloroform and ethylacetate. The ratio of internal water volume to solvent volume was varied from 0.01 up to 0.48. The ratio of PLG to solvent concentration was varied between 11 and 217.

Stabilizer: The stabilizer in the process medium was varied between carboxymethylcellulose (CMC), polyvinyl alcohol (PVA) and mixtures of CMC and PVA. The content of the stabilizer in the process medium as varied between 1% and 5%. A cationic lipid (DOTAP) was added to the stabilizer.

Mixing Conditions: Both 30 ml syringes and 20 ml syringes were tested as the mixing vessel for the primary emulsion. Various mixing heads on the Silverson mixer were also tested.

Summary

A quick release, high efficiency, porous, 1-10 μm DNA microsphere formulation was developed and tested. CTL responses to two antigens, Her-2/neu and TbH9, were generated using these DNA microspheres. Moreover, T cells generated by Her-2/neu or TbH9 DNA immunizations have been shown to recognize and kill human tumors expressing the corresponding antigen. Intramuscular and intraperitoneal routes proved best for CTL elicitation. Several AGPs provided substantial CTL adjuvant activity to the DNA microspheres. Sodium chloride inhibited CTL generation to DNA microspheres.

### Example 2

AGP Adjuvants Enhance Efficacy of DNA Microspheres

In this example, 10 μg of AGP in aqueous formulation was added to 10 μg DNA encapsulated in PLG microspheres in suspension in PBS. The microspheres were prepared with a 503H polymer using a double emulsion technique, CMC stabilizer and the “5.1” method, resulting in microspheres of about 1 to 10 μm in diameter. The microspheres were injected i.m. in groups of four C57B1/6 mice. Spleens were harvested 3-4 weeks following immunization and processed into single cell suspensions. Splenocytes were stimulated in vitro with EL-4 cells stably expressing the TbH9 gene. CTL activity was assayed by standard protocols. Fresh splenocytes were also stimulated in vitro with 5 μg/ml recombinant TbH9, and supernatants assayed for IFN-gamma secretion, by ELISA. The results demonstrate that AGP adjuvants can provide strong cellular immune responses to an antigen encoded by DNA encapsulated in microspheres, superior to that occurring without adjuvant.

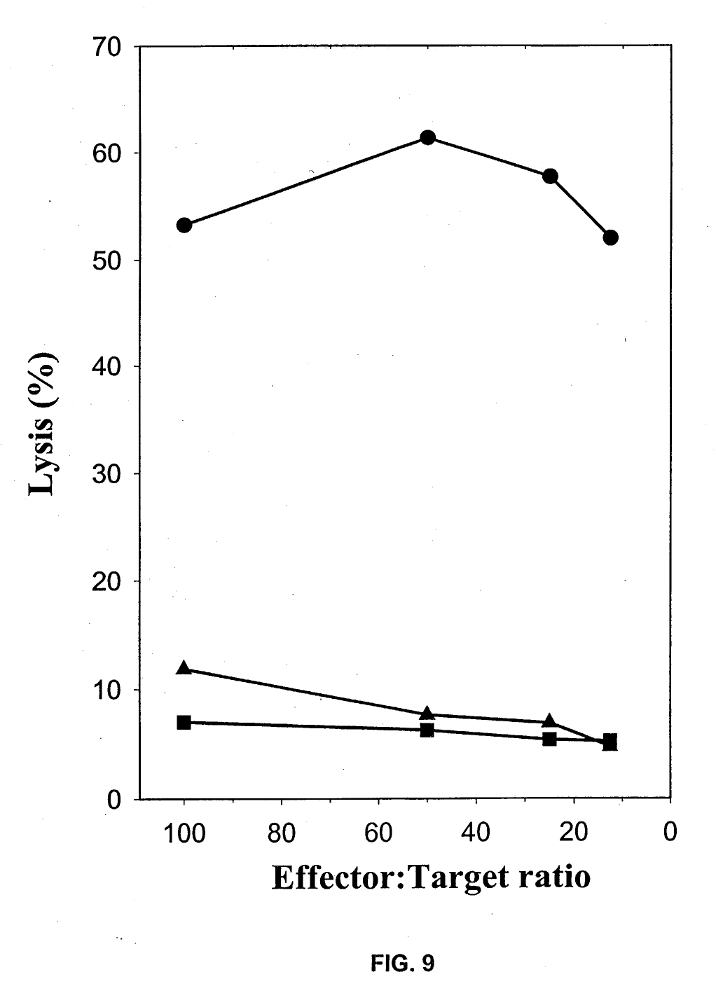

FIG. 10 is a bar graph showing IFN-gamma secretion (in pg/ml) in response to in vitro stimulation with recombinant TbH9, assayed using splenocytes harvested from mice 3-4 weeks following immunization with TbH9 DNA encapsulated in PLG microspheres with AGP.

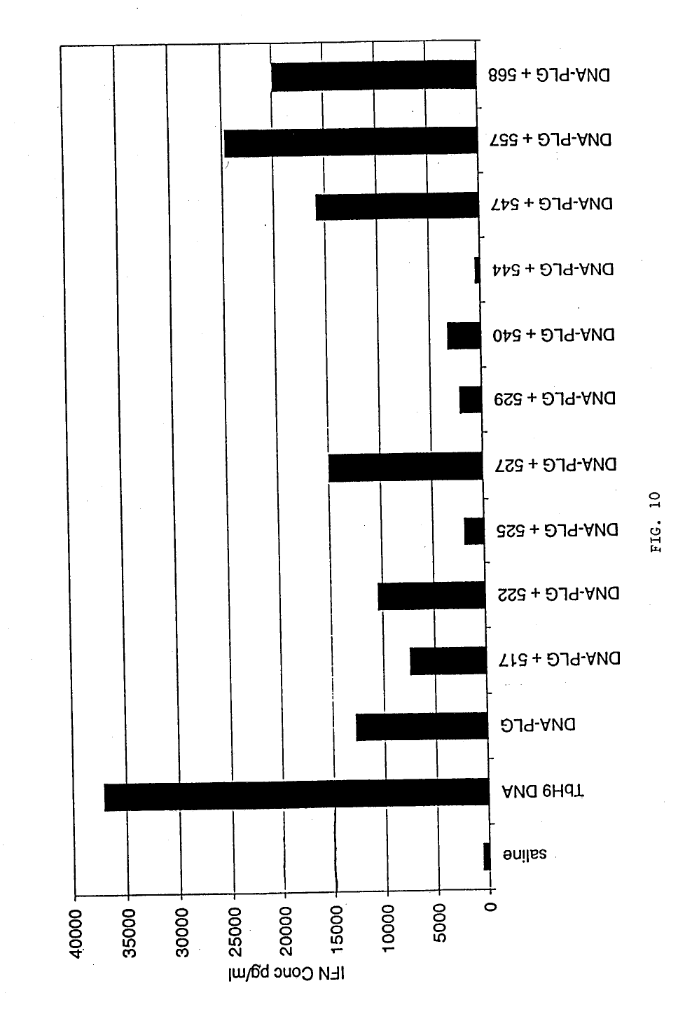

FIG. 11 is a graph showing mean CTL activity after a single in vitro stimulation with EL-4 cells stably expressing the TbH9 gene of splenocytes harvested from mice immunized with TbH9 DNA encapsulated in PLG microspheres to which AGP was added. The graph shows mean specific lysis as a function of effector:target ratio for immunization conditions including saline (closed diamonds), naked DNA (dark squares), DNA-PLG (lower triangles), and DNA-PLG plus AGP-517 (light X's), 522 (asterisks), 525 (circles), 527 (+'s), 529 (dashed line), 540 (−'s), 544 (open diamonds), 547 (light squares), 557 (upper triangles), or 578 (dark X's).

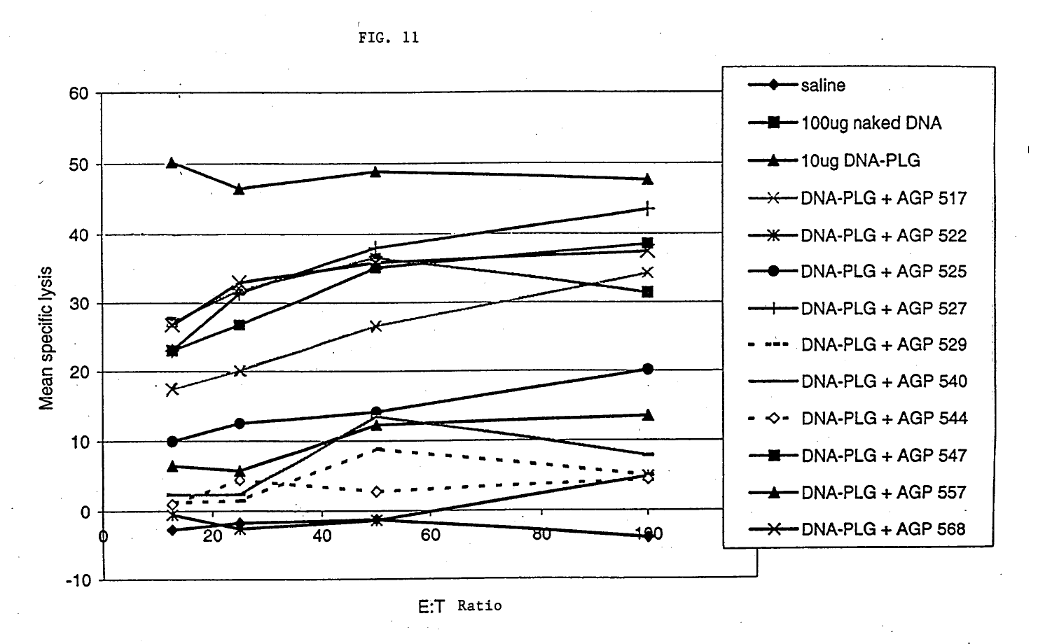

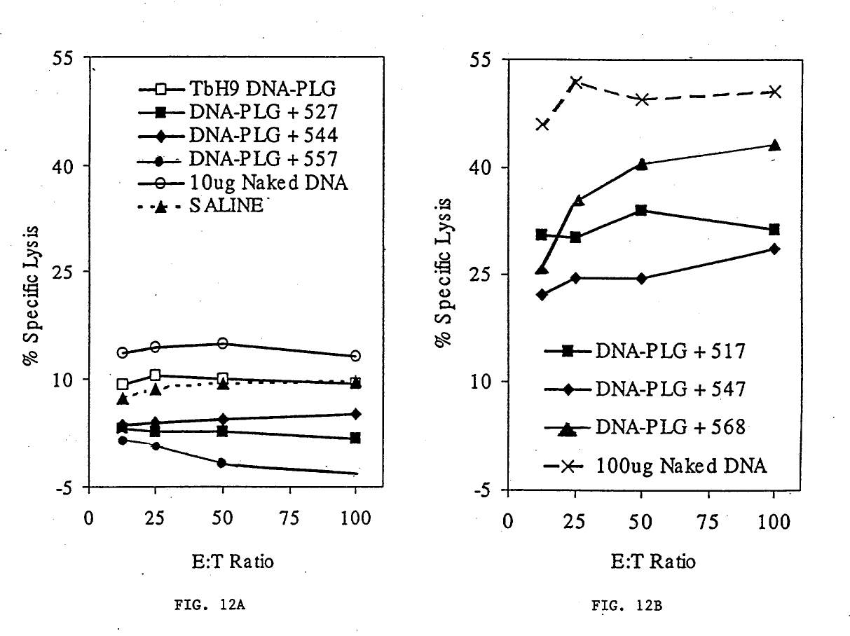

FIG. 12A shows mean CTL activity after a second in vitro stimulation of splenocytes from mice immunized with TbH9 DNA-PLG alone (open squares), with AGP-527 (closed squares), 544 (dark diamonds), 557 (closed circles), or with naked DNA (open circles) or saline (triangles).

FIG. 12B shows mean CTL activity after a second in vitro stimulation of splenocytes from mice immunized with TbH9 DNA-PLG with AGP-517 (closed squares), 547 (dark diamonds), 568 (light triangles), or with naked DNA (X's).

### Example 3

Immune Responses Elicited in Monkeys By Encapsulated DNA

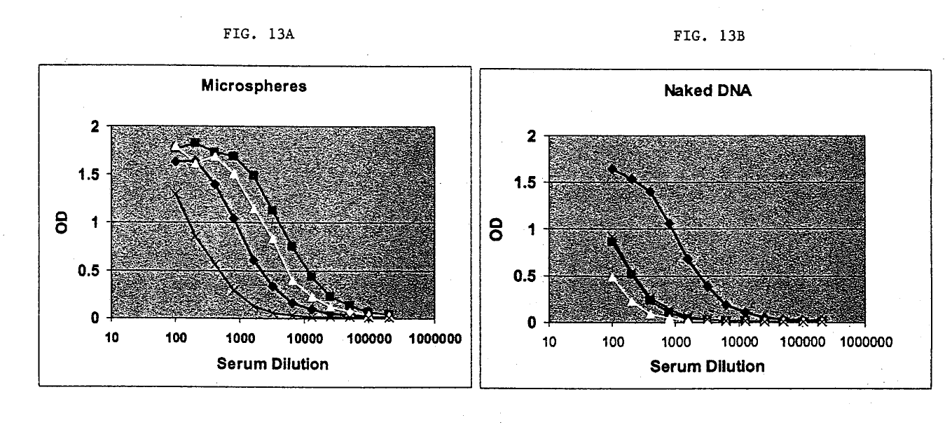

This example describes the immune responses elicited in Rhesus macaques following three immunizations, at monthly intervals, with either naked TbH9-VR1012 DNA or TbH9-VR1012 DNA encapsulated in microspheres that were prepared in accordance with the invention. Naked DNA consisted of 3.3 mg plasmid +40 μg RC 527-AF, immunized by intradermal and intramuscular routes. Microsphere DNA consisted of 3 mg plasmid +50 μg RC 568-AF delivered intramuscularly. There were four animals in each group. The results, shown in FIGS. 13-15, demonstrate that the microsphere-encapsulated DNA elicited stronger immune responses than were observed with naked DNA.

FIGS. 13A-B are graphs showing serum antibody titers to TbH9 of Rhesus macaques four weeks after a 3^{rd} immunization with TbH9, encapsulated in microspheres and administered intramuscularly (FIG. 13A), or delivered as naked DNA via intradermal or intramuscular routes (FIG. 13B).

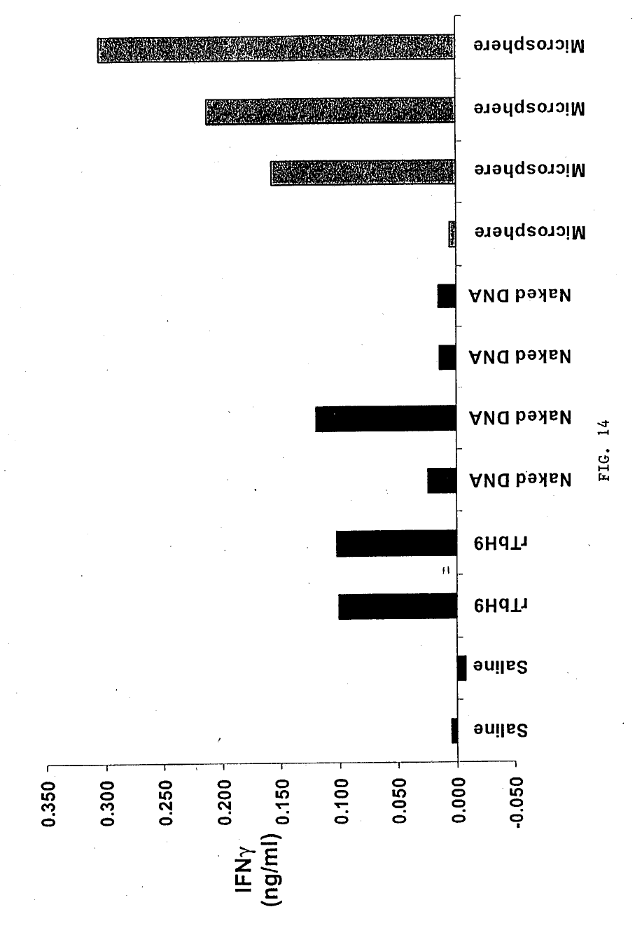

FIG. 14 is a bar graph showing antigen-induced gamma interferon (IFN-γ) production from monkey PBMC at 4 weeks after a 3^{rd} immunization with saline, recombinant TbH9 (rTbH9), naked DNA encoding TbH9 or microspheres encapsulating DNA encoding TbH9. Individual bars represent individual subjects.

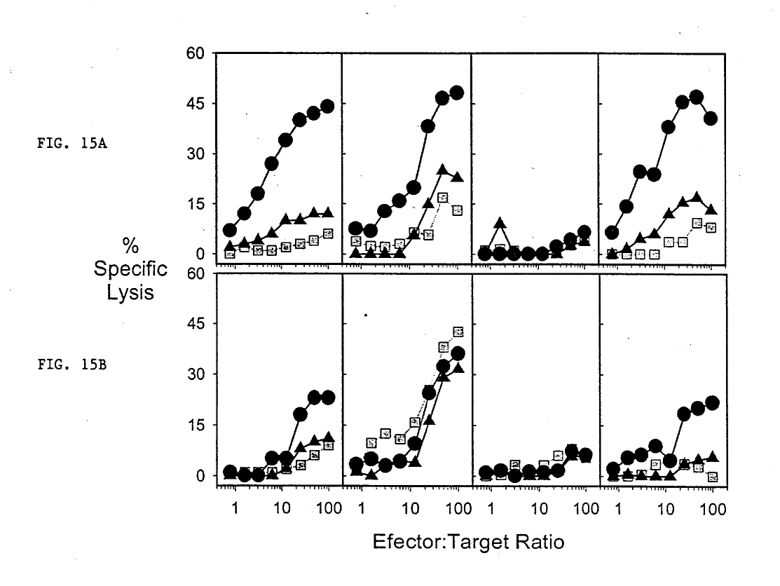

FIGS. 15A-B are graphs showing monkey CTL response at two months after a 3^{rd} immunization with microencapsulated (FIG. 15A) or naked (FIG. 15B) DNA encoding TbH9. Percent specific lysis is plotted as a function of effector:target ratio.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A nucleic acid delivery system comprising deoxyribonucleic acid (DNA) encapsulated in biodegradable polymeric microspheres, wherein at least 50% of the DNA comprises supercoiled DNA, and wherein at least 50% of the DNA is released from the microspheres within about 7 days at about 37° C.

2. The nucleic acid delivery system of claim 1, wherein the microspheres have an encapsulation efficiency of at least about 40%.

3. The nucleic acid delivery system of claim 1, wherein at least 70% of the DNA is released from the microspheres within about 7 days at about 37° C.

4. The nucleic acid delivery system of claim 1, wherein at least 50% of the DNA is released from the microspheres within about 4 days at about 37° C.

5. The nucleic acid delivery system of claim 1, wherein at least 90% of the microspheres are about 1 to about 10 μm in diameter.

6. The nucleic acid delivery system of claim 1, wherein the microspheres comprise poly(lacto-co-glycolide) (PLG).

7. The nucleic acid delivery system of claim 6, wherein the PLG has a molecular weight of from about 8 kDa to about 65 kDa.

8. The nucleic acid delivery system of claim 6, wherein the PLG includes ester end groups or carboxylic acid end groups.

9. The nucleic acid delivery system of claim 1, further comprising an adjuvant.

10. The nucleic acid delivery system of claim 9, wherein the adjuvant comprises an aminoalkyl glucosaminide 4-phosphate (AGP).

11. The nucleic acid delivery system of claim 9, wherein the adjuvant comprises MPL.

12. The nucleic acid delivery system of claim 1, wherein the DNA encodes an antigen associated with cancer or infectious disease.

13. The nucleic acid delivery system of claim 12, wherein the cancer is breast cancer.

14. The nucleic acid delivery system of claim 13, wherein the antigen is her2/neu.

15. The nucleic acid delivery system of claim 12, wherein the infectious disease is tuberculosis.

16. The nucleic acid delivery system of claim 15, wherein the antigen is TbH9.

17. A nucleic acid delivery system comprising DNA encapsulated in biodegradable polymeric microspheres, wherein at least about 70% of the DNA comprises supercoiled DNA, and wherein at least about 70% of the DNA is released from the microspheres within about 7 days at about 37° C.

18. A method for encapsulating nucleic acid molecules in microspheres comprising:
(a) dissolving a polymer in a solvent to form a polymer solution;
(b) adding an aqueous solution containing nucleic acid molecules to the polymer solution to form a primary emulsion;
(c) homogenizing the primary emulsion;
(d) mixing the primary emulsion with a process medium comprising a stabilizer to form a secondary emulsion; and
(e) extracting the solvent from the secondary emulsion to form microspheres encapsulating nucleic acid molecules;
wherein the nucleic acid molecules are maintained above freezing temperature and below 37° C. during steps (b)-(d); wherein at least 50% of the nucleic acid molecules retain a supercoiled formation through the extraction step; and wherein the microspheres release at least about 50% of the nucleic acid molecules within about 7 days at 37° C.

19. The method of claim 18, wherein the encapsulation efficiency is at least about 40%.

20. The method of claim 18, wherein the microspheres release at least about 50% of the nucleic acid molecules within about 4 days.

21. The method of claim 18, wherein at least about 90% of the microspheres are from about 1 μm to about 10 μm.

22. The method of claim 18, wherein the polymer comprises PLG.

23. The method of claim 22, wherein the PLG includes ester end groups or carboxylic acid end groups.

24. The method of claim 22, wherein the PLG has a molecular weight of from about 8 kDa to about 65 kDa.

25. The method of claim 18, wherein the nucleic acid molecules are maintained at about 2° C. to about 35° C. prior to the extraction.

26. The method of claim 18, wherein the nucleic acid molecules are maintained at about 4° C. to about 25° C. prior to the extraction.

27. The method of claim 18, wherein the solvent comprises dichloromethane, chloroform, or ethylacetate.

28. The method of claim 18, wherein the polymer solution further comprises a cationic lipid.

29. The method of claim 18, wherein the polymer solution further comprises an adjuvant.

30. The method of claim 29, wherein the adjuvant comprises MPL.

31. The method of claim 18, wherein the stabilizer comprises carboxymethylcellulose (CMC), polyvinyl alcohol (PVA), or a mixture of CMC and PVA.

32. The method of claim 31, wherein the stabilizer further comprises a cationic lipid.

33. The method of claim 18, wherein the stabilizer comprises from about 1% to about 5% of the process medium.

34. The method of claim 18, wherein the solvent comprises an internal water volume of from about 0.001% to about 0.5%.

35. The method of claim 18, wherein the aqueous solution comprises an ethanol content of from about 0% to about 75% (v/v).

36. The method of claim 18, wherein the nucleic acid molecule comprises DNA.

37. The method of claim 36, wherein the aqueous solution comprises about 0.2 to about 12 mg/ml DNA.

38. The method of claim 36, wherein the DNA comprises a plasmid of about 3 kb to about 9 kb.

39. The method of claim 18, wherein the aqueous solution further comprises an adjuvant.

40. The method of claim 39, wherein the adjuvant comprises QS21.

41. The method of claim 18, wherein the aqueous solution further comprises a stabilizer.

42. The method of claim 41, wherein the stabilizer comprises bovine serum albumin.

43. A composition comprising nucleic acid molecules encapsulated in microspheres produced by the method of claim 18.

44. A method for delivering a nucleic acid molecule to a subject comprising administering to the subject a nucleic acid delivery system of claim 1.

45. A method for eliciting an immune response to an antigen in a subject comprising administering to the subject a nucleic acid delivery system of claim 12.

46. A method for treating or preventing a cancer associated with her2/neu antigen in a subject comprising administering to the subject a therapeutically effective amount of a nucleic acid delivery system of claim 14.

47. A method for treating or preventing tuberculosis in a subject comprising administering to the subject a therapeutically effective amount of a nucleic acid delivery system of claim 16.

48. An adjuvant for enhancing the immunostimulatory efficacy of microspheres encapsulating nucleic acid molecules comprising an aminoalkyl glucosaminide 4-phosphate (AGP).

49. The adjuvant of claim 48, wherein the AGP comprises an aqueous formulation.

50. The adjuvant of claim 52, wherein the AGP comprises 517, 527, 547, 557 or 568.

51. A method for enhancing the immunostimulatory efficacy of microspheres encapsulating nucleic acid molecules comprising administering an AGP as an adjuvant to administration of microspheres encapsulating nucleic acid molecules, wherein the AGP enhances the immunostimulatory efficacy of the microspheres.

52. The method of claim 51, wherein the AGP is administered simultaneously with the microspheres.

53. The method of claim 51, wherein the AGP is administered before or after administration of the microspheres.
